(19) United States
(12) Patent Application Publication
(10) Pub. No.: US 2005/0096597 A1
(43) Pub. Date: May 5, 2005

(54) Safety shield system for a syringe

(75) Inventors: Jamie Crawford, New York, NY (US); Roger Groskopf, Saddle Brook, NJ (US); Mark C. Newby, Tuxedo, NY (US)

Correspondence Address: COHEN, PONTANI, LIEBERMAN & PAVANE, 551 FIFTH AVENUE, SUITE 1210, NEW YORK, NY, 10176, US

(73) Assignee: Becton, Dickinson and Company

(21) Appl. No.: 10/699,953

(22) Filed: Nov. 3, 2003

(51) Int. Cl. A61M 5/32

(52) U.S. Cl. 604/198

(57) ABSTRACT

A medical device for delivering a medicament to a patient includes a syringe assembly having a syringe barrel, a needle cannula coupled to the syringe barrel, and a plunger having an end connected to a stopper for insertion into an end of the barrel. A shield system is provided for preventing inadvertent needle sticks after use of the syringe. The shield system includes an actuator mounted on the syringe barrel and movable from an initial position to a retracted position in response to the application of the medicament delivery force. A shield arranged on the front portion of the barrel is responsive to the position of the actuator to deploy the shield from a first position wherein the tip of the needle cannula is exposed, to a second position wherein the shield covers the needle cannula tip.

### BACKGROUND OF THE INVENTION

1. Field of the Invention

The present invention relates to a medical device for delivering a dose of medicament by injection and having a shield system for preventing accidental needle sticks after use. More particularly, the present invention is directed to a syringe assembly including a safety shield system.

2. Description of the Related Art

Syringes used for the delivery of medicaments to patients are well known. Oftentimes syringes are prefilled with a dosage of a medicament or other substance by a pharmaceutical manufacturer and then distributed to end users such as health care professionals or patients for administration of the prefilled medicament. Such syringes typically include a cylindrical hollow barrel which may be formed of a glass or plastic material and which includes the medicament. One end of the barrel is fitted with a fixed or removable hollow needle, and the other end of the barrel receives a plunger having a stopper which is slidable with respect to the barrel for delivery of the medicament to the hollow needle, i.e., to urge the medicament toward and out of the needle. A syringe assembly, which typically includes the above-described components, is usually stored with a removable needle cover which protects the needle from damage during storage and handling. Prior to use, the needle cover is removed to expose the needle.

To prevent a syringe user and, in particular, a health care provider from inadvertent sticks by the needle after use of the syringe on a patient, the syringe assembly may incorporate a safety shield which forms a guard over the needle after use. Certain attributes to be considered in such syringe assemblies are that the assemblies should be intuitive and easy to use, should be passive so that 100% shield deployment occurs, and should provide for one-hand operation. Other attributes are that such syringe assemblies require no change in current medicament delivery techniques, allow for dose adjustment, are autoclavable, and allow for the inspection of contents before and after activation of the shield. Moreover, the use of the shield must not detrimentally affect processing and loading of the syringe at the pharmaceutical company, the assembly must be easy to manufacture, must prevent accidental activation, and must limit the possibility of incurring cosmetic or structural damages.

### SUMMARY OF THE INVENTION

The present invention relates to a syringe assembly incorporating a safety shield for covering the needle of the syringe assembly after administration of a dosage of medicament. The safety shield is automatically activated upon the application of medicament delivery pressure to the syringe.

According to the present invention, a syringe assembly medical device for delivering a medicament to a patient includes a syringe having a syringe barrel defining a reservoir within which a medicament may be held; the syringe barrel having a front end and a rear end. A needle or needle cannula (those terms being used interchangeably herein) is provided proximate the front end of the barrel and is in fluid communication with the reservoir. A shield system for preventing inadvertent needle sticks after use of the syringe assembly includes a shield actuator defining a hollow shield body in which the syringe barrel is disposed and movably mounted. A shield is arranged on the front end of the syringe barrel and releasably connected thereto by a retention device. Upon activation of the shield actuator, the retention device releases the shield from the front end of the syringe barrel, whereupon the shield is movable under the influence of an urging member from a first position wherein the needle cannula is exposed, to a second position wherein the tip of the needle cannula is covered by the shield. The urging member, by way of non-limiting example, may be a coil spring. Activation of the actuator occurs upon commencement of use of the medical device. The shield, urging member, and the retention device are arranged at the front end of the barrel.

The syringe assembly includes a plunger having a first end with a stopper inserted in the syringe barrel. The second end of the plunger has a thumb pad or thumb press area for receiving medicament delivery pressure for pressing the plunger into the syringe barrel to deliver the medicament. The terms “thumb pad” and “thumb press area” are used interchangeably herein and designate a region coupled to or otherwise formed on an end of the plunger and which may be depressed by the thumb or finger of a user during use of the medical device.

The syringe barrel may comprise a cylindrical barrel portion for holding the medicament and a front portion arranged proximate the front end of the syringe barrel for coupling to the cannula and the shield. The syringe barrel may be made of a glass or a plastic material. Alternatively, the cylindrical barrel portion of the syringe barrel may be made of glass and the front portion of the syringe barrel may be made of plastic.

The retention device may comprise a catch element arranged on the front end of the barrel which is engageable with a retention element on the shield. The catch element engages the retaining element for preventing the shield from moving toward the second position under the influence of an urging member. The retaining element may be mounted on a flexible arm formed on the shield.

The release mechanism may include a cam arranged on an inner surface of the actuator and positioned to move the flexible arm to release the engagement between the shield and the retention device when the actuator is moved to the retracted position. The cam may further comprise a locking surface for engaging the shield when the shield is in the second position for preventing the shield from moving from the second position back toward the first position.

The flexible arm holding the retaining element may be arranged on the shield and the catch may be connected proximate the front end of the syringe barrel, or vice versa.

The medical device may further comprise an actuator locking device arranged on the barrel for retaining the actuator in the retracted position. The locking device comprises an actuator catch arranged on the actuator and a projection on the syringe barrel. A removable clip is connectable to the syringe barrel for preventing movement of the actuator to the retracted position while the clip is connected to the syringe barrel.

The shield may include a radially outward extending pin which is received in a slot defined in the actuator for guiding movement of the shield from the first position to the second position. An end of the slot retains the pin of the shield at the second position when the shield is moved to the second position.

The present invention allows one-hand operation and requires no change in current medicament delivery techniques. Since the entire shield assembly is arranged in front of the medicament holding portion of the syringe, the present invention allows dose adjustment, inspection of contents after activation of the shield, and allows the drug and scale on the syringe to be visible to the user. Activation of the shield is not detrimental to the administration of the dose because it is activated prior to delivery of the dose.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings, wherein like reference characters denote similar elements throughout the several views:

FIG. 1 is a perspective cross-sectional view of a medical device according to the present invention;

FIG. 2 is an exploded perspective view of the medical device of FIG. 1;

FIG. 3 is a perspective cross-sectional view of a syringe barrel of the medical device shown in FIG. 1;

FIG. 3a is a partial perspective view of a further embodiment of a syringe barrel according to the invention;

FIG. 4 is a perspective cross-sectional view of a shield of the medical device of FIG. 1;

FIG. 4a is a cross-section side view of a further embodiment of a shield according to the present invention;

FIG. 5 is a perspective cross-sectional view of an actuator of the medical device of FIG. 1;

FIG. 6 is a side cross-sectional view of the medical device of FIG. 1 in a state prior to use;

FIG. 7 is a side cross-sectional view of the medical device of FIG. 6 with the cap removed;

FIG. 8 is a side cross-sectional view of the medical device of FIG. 7 in an activatable state;

FIG. 9 is a side cross-sectional view of the medical device of FIG. 8 when medicament delivery pressure is applied to the plunger rod;

FIG. 10 is an enlarged side cross-sectional view of the medical device of FIG. 9 in which the shield is urged against a patient's skin;

FIG. 11 is an side cross-sectional view of the medical device of FIG. 1 after delivery of the full dosage and after the device is removed from a patient;

FIG. 12 is a cross-section of the medical device along line XII-XII in FIG. 6;

FIG. 13 is a side cross-sectional view of another embodiment of the medical device according to the present invention;

FIG. 14 is a side cross-sectional view of the medical device of FIG. 13 with the shield in the activated position; and

FIG. 15 is a side cross-sectional view of the medical device similar to FIG. 13 and showing an alternative embodiment.

### DETAILED DESCRIPTION OF THE PRESENTLY PREFERRED EMBODIMENTS

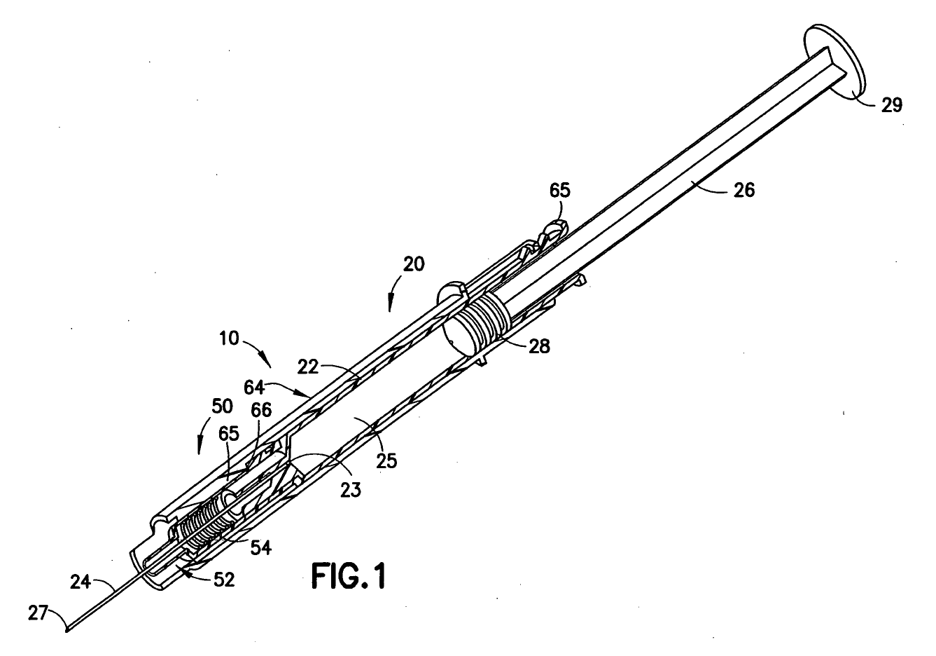

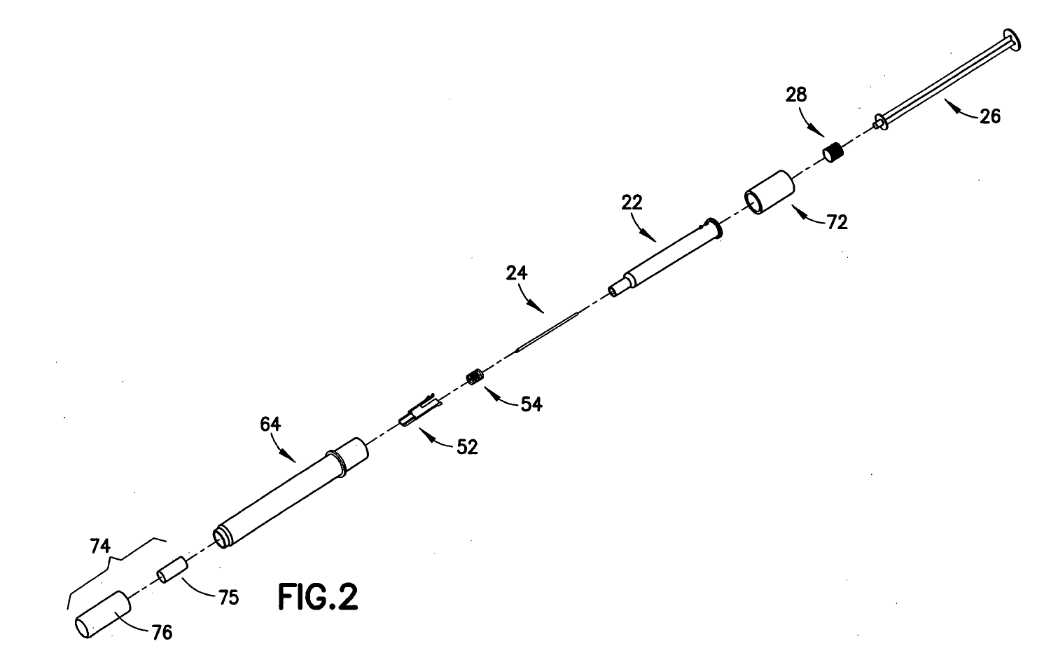

FIGS. 1-2 show a medical device 10 according to the present invention for delivery of a medicament into a patient constructed in accordance with an embodiment of the present invention. As used herein, the term “medicament” is intended to refer to any drug substance, vaccine, or other liquid substance that is injected into a patient. The medical device 10 includes a syringe 20 which can be prefilled with the medicament to be delivered, and a shield system 50 which surrounds the syringe.

The syringe assembly includes a barrel 22 having an axis “A” and defining a reservoir 25 within which the medicament may be held prior to use of the medical device 10. The syringe 20 also includes a needle cannula 24 having a rearward end 23 and a forward tip 27. The rearward end is in fluid communication with the reservoir 25. The needle cannula 24 may be permanently connected to a front end of the barrel 22 using an adhesive, glue, or other known or hereafter developed material or technique, or it may be detachable from the barrel 22 such as, for example, using a luer-type connection. As further shown in FIGS. 1 and 2, a removable needle shield 74 is disposed over the needle cannula 24 on the front end of the barrel 22 to protect the needle from damage during handling of the syringe assembly, and to protect users from being stuck by the needle prior to its intended use. The needle shield 74 preferably includes a pliable part 75 and a rigid part 76.

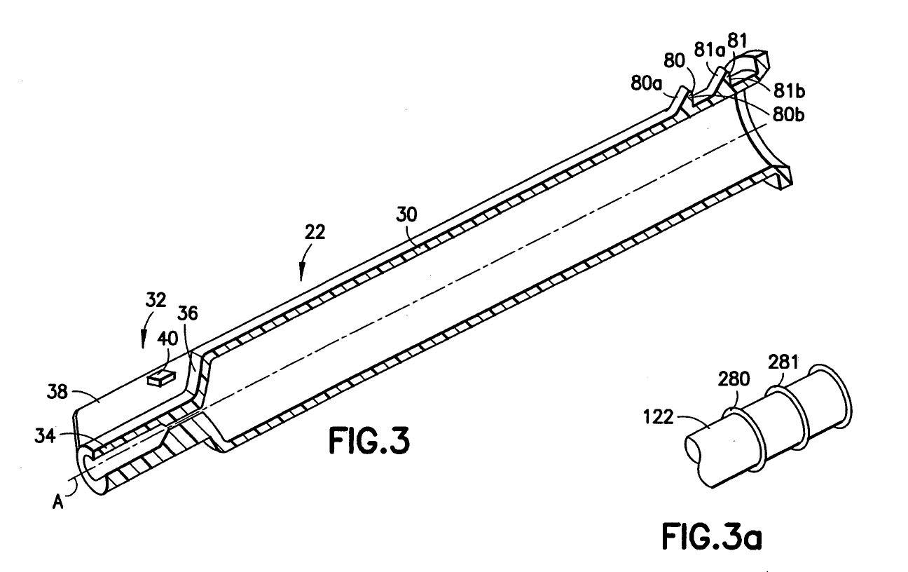

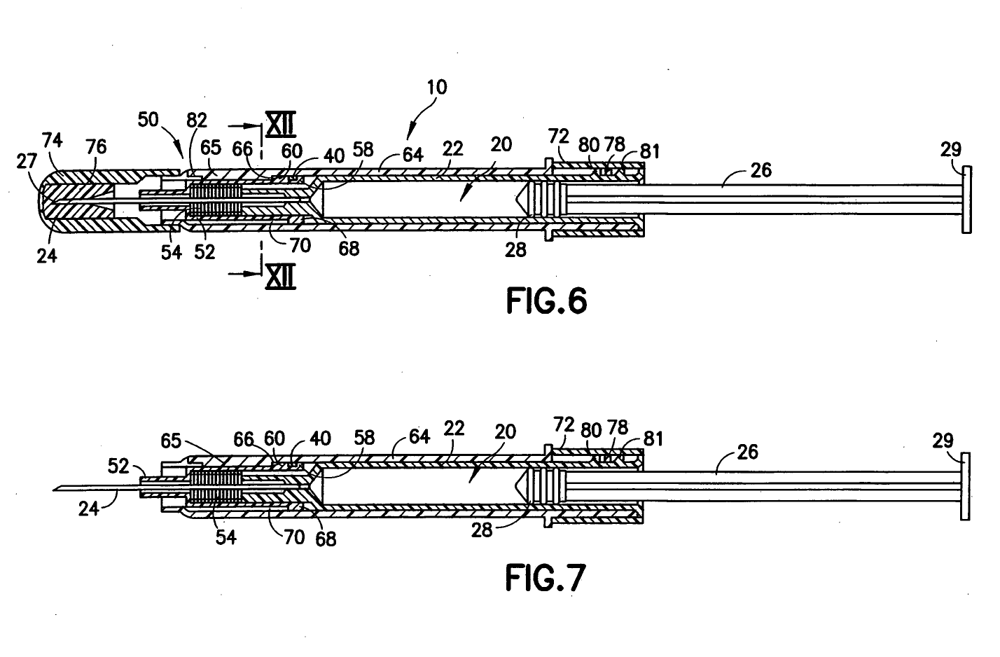

As shown in FIG. 3, the syringe barrel 22 includes a cylindrical portion 30 for defining the reservoir, and a front portion 32 connected to the cylindrical portion 30. The front portion 32 has a small cylinder region 34 for receiving the rearward end 23 of the cannula 24, a transition section 36 connecting the small cylinder region 34 to the cylindrical portion 30, and a web 38 having a catch or protuberance 40 which will be described in more detail below. A first projection 80 and a second projection 81 are arranged proximate a rear end of the barrel 22 at which a rim 65 is formed. As shown, an initial channel 83 is defined between projections 80 and 81, and a retracted channel is defined between projection 81 and rim 65. The syringe barrel 22 may be made entirely of a plastic material or a glass material, but a plastic material is preferred for integrally forming the components of the barrel 22, such as the catch 40 with the cylindrical portion 30. Alternatively, the cylindrical portion 30 may be made of glass and the front portion 32 made of plastic attached to the front of the cylindrical portion 30 by adhesive, glue, or welding. A plunger rod 26 (see also FIGS. 1 and 6) has a first end inserted in the barrel 22 with a stopper or piston 28 arranged on the first end and movable with the plunger rod 26 within the barrel 22. A second end of the plunger rod 26 includes a thumb pad 29 used for receiving pressure from the user's thumb, forefinger, etc. for moving the piston 28 into and within the barrel 22 and along axis “A” to deliver the medicament through the needle cannula 24.

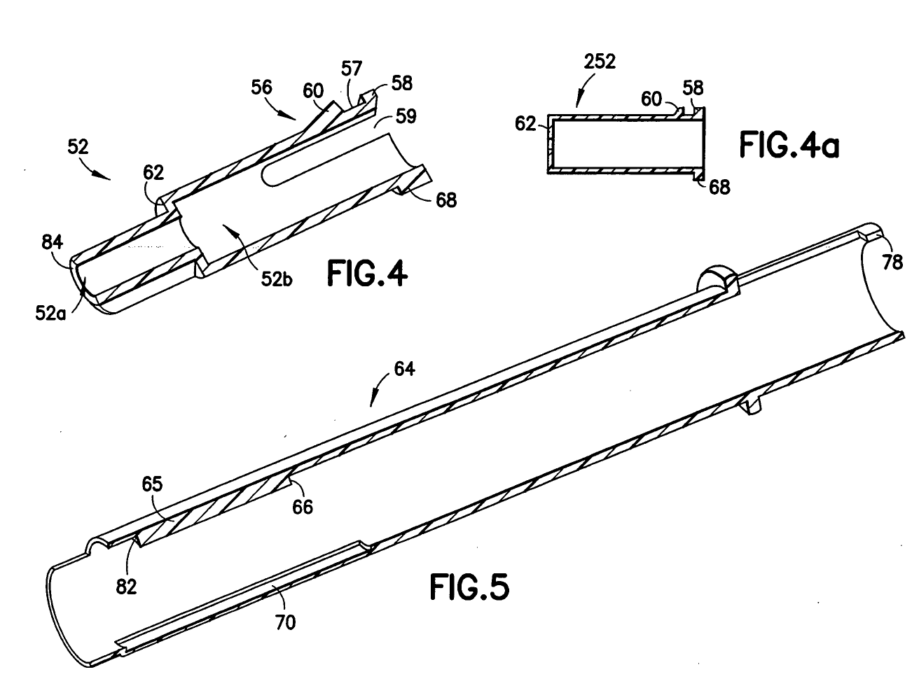

The shield system 50 includes a hollow shield body 52 which is connectable to the front portion 32 of the syringe barrel 22. As shown in FIG. 4, a step 62 divides the shield 52 into a first portion 52a having a first diameter and a second portion 52b having a second diameter that is different, and preferably larger, than the first diameter. An urging member such as a spring 54 (FIG. 1) or elastic arm is arranged between the barrel 22 and the step 62 for urging the shield away from the front of the barrel 22 along axis “A”. The shield 52 includes a slot 59 disposed in a sidewall for forming a lever arm 56 having two spaced-apart projections including a retaining element 58 and a release element 60 such that a notch or seat 57 is formed therebetween. The notch 57 is positioned on the flexible arm 56 such that catch 40 of the syringe barrel 22 seats in the notch when the shield 52 is fully positioned on the front portion of the barrel 22 with the web 38 seated in the slot 59. The engagement of the retaining element 58 with the catch 40 prevents deployment of the shield 52 by a force provided by the urging member 54 in a direction away from the front end of the barrel 22 along axis “A”. Thus, to position the shield about the front portion 32 of the barrel 22, the urging member 54 is placed between the step 62 and the front portion 32 and the shield 52 is oriented so that the slot 59 is aligned with the web 38, whereupon the shield is pushed back onto the front portion 32 against the force of the urging member 54. By applying slight pressure to lever arm 56 in a direction to narrow the slot 59, the catch 40 can be positioned, and seat, within notch 57 to anchor the shield 52 to the front portion 32 against the force of the urging member 54. In this position, the urging member 54 is in a “charged” state and the tip of the cannula 24 extends past the forward end 84 of the shield 52 so that the medical device may be used to pierce or otherwise enter a patient for delivery of the medicament. As described below, upon release of the catch 40 from the seat 57 the shield 52 is movable from its first position (wherein the catch 40 is seated in notch 51 and the tip 27 of the needle cannula is exposed), to an activated or deployed second position in which the shield 52 covers the tip 27 of the needle cannula 24 (FIG. 11). The shield 52 includes a pin 68 which guides movement of the shield along the front portion 32 of the barrel 22 from the first position to the activated second position as described below.

In an alternative embodiment shown in FIG. 4a, the shield 252 may be designed so that the step 62 is arranged at the front end of the shield. In this embodiment, there is no small diameter portion 52a as shown in FIG. 4 and the size of the urging member 54 must be adjusted to provide the proper amount of energy to push the shield 252 to the activated position.

With reference to FIGS. 1, 2 and 5, the shield system 50 also includes an actuator 64 which is arranged on the barrel 22 for movement from an initial starting position (shown in FIG. 1) to a retracted position. The actuator 64 is preferably molded from transparent polystyrene so that the content of the barrel 22 is viewable. Other suitable transparent materials which may be used to form the shield body include acrylic, PET, or polycarbonate materials. Before use of the medical device 10, the actuator 64 is in the starting position as shown in FIG. 1 and a removable storage or packaging clip 72 (see FIG. 2) is arrangeable on the syringe barrel 22 to prevent premature or unintended activation of the actuator 64. A cam 65 having a cam face 66 is arranged on the actuator 64. As the actuator moves toward the retracted position in a direction away from needle cannula 24 (with the packaging clip 72 removed from the barrel 22), the cam face 66 interacts with the release element 60 arranged on the flexible arm 56 of the shield 52 and moves the flexible arm 56 to narrow the slot 59 and to release the engagement of the catch 40 from the notch 57. The cam 65 of the actuator 64 also includes a locking face 82 for engaging the retaining element 58 when the shield 52 is in the deployed second position shown in FIG. 6 and described below. The actuator 64 further includes a guide track 70 for receiving a pin 68 arranged on the shield 52 for maintaining alignment of the shield 52 with respect to the needle cannula 24 as the shield is moved to its second position.

The actuator 64 includes an actuator catch 78. When the actuator is in its starting position the catch seats in the initial channel 83, and when the actuator is in its retracted position, the catch 78 seats in the retracted channel 85. Each of the projections 80, 81 has an angled front face 80a, 81a, respectively, and a straight rear face 80b, 81b, respectively. The angled front face 81a facilitates movement of the catch 78 from the initial channel 83 to the retracted channel 85. The straight face 80b prevents movement of the actuator catch 78 out of the initial channel 83 toward the needle cannula 24, and the straight face 81b maintains actuator catch 78 in the retracted channel 85. Instead of projections 80, 81, the barrel 22 may comprise annular ribs 280, 281 as shown in FIG. 3a.

FIG. 6 shows the medical device according to the present invention in a state in which it is stored prior to its intended use. In this state, the shield 52 is fully inserted over the front of the syringe barrel 22 and the removable cap 74 is placed over the needle 24. The retaining element 58 on the shield 52 is engaged behind the catch 40 on the syringe barrel 22, thus preventing the urging member 54 from pushing the shield 52 forward. The actuator 64 is in the starting position and is prevented from moving toward the retracted position by the packaging clip 72. As explained above, the packaging clip 72 prevents inadvertent activation of the safety shield during handling of the syringe assembly prior to use.

As shown in FIG. 12, a slot 90 is defined in an inner surface of the actuator for receiving a free end of the web 38 which is connected to the barrel 22. This maintains the alignment of the actuator 64 with the features of the barrel such as the blocking projections 80, 81. Furthermore, FIG. 12 also shows that the small cylinder 34 of the front portion 32 of the barrel 22 has a flattened area 92 which provides clearance for the release movement of the lever arm 56 of the shield 52.

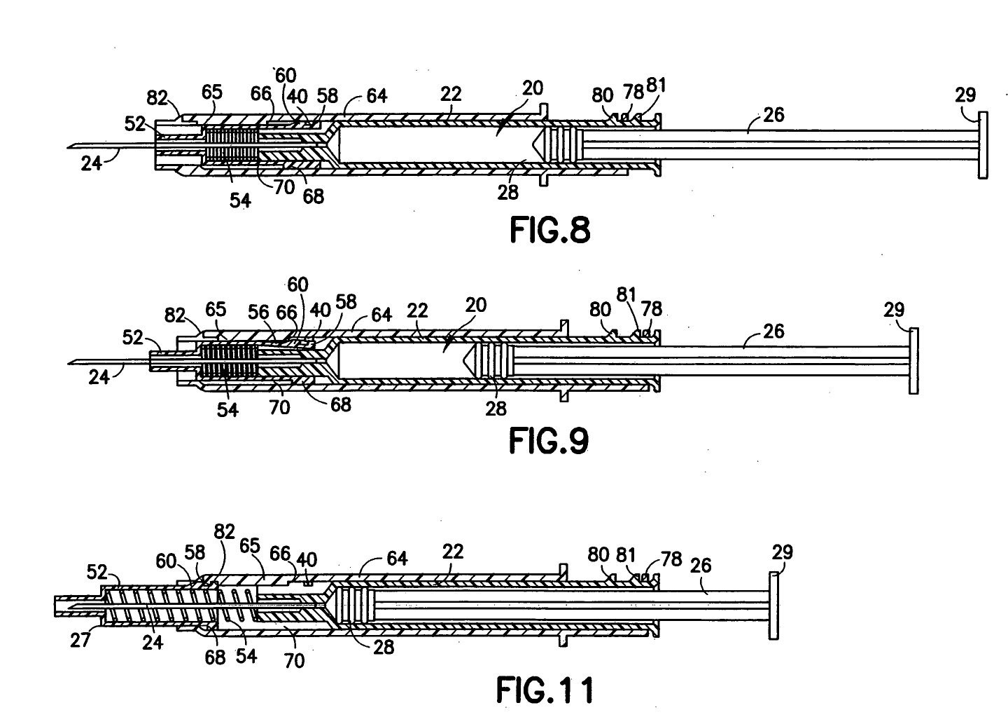

When the medical device 10 is ready to be used, the cap 74 is removed as shown in FIG. 7. In this state, the packaging clip 72 remains on the barrel 22 so that the plunger rod 26 may be moved into or out of the barrel 22 to adjust the dosage in the barrel without activating the shield 52. After the dosage has been properly adjusted and is ready to be delivered, the packaging clip 72 is removed and the needle may be inserted in the patient to deliver the dosage of medicament (see FIG. 8). The force required to depress the plunger rod 26 into the barrel 22, i.e., to move the piston 28 in the barrel 22, is greater than the force required to move the actuator 64 on the barrel 22 to the retracted position. Accordingly, at the commencement of applying medicament delivery pressure to the thumb pad 29 the actuator 64 first moves to the retracted position wherein activator catch 78 seats in retracted channel 85. This operation is achieved by dimensioning the actuator 64 to easily pass over the outer surface of the barrel 22 with a minimal clearance and by designing the inclined leading edge 81a of blocking projection 81 and actuator catch 78 such that only a small force is required to move the actuator catch 78 over the blocking projection 81.

As the actuator 64 moves to the retracted position, the cam face 66 of the actuator pushes on the release element 60, thereby bending the flexible arm 56 radially inward to disengage the retaining element 58 from the catch 40 (see FIG. 9). When the actuator 64 is in the retracted position as shown in FIG. 9, the shield 52 is no longer prevented from moving forward by the catch 40. Accordingly, the shield 52 moves under the urgency of the urging device 54 toward the activated position.

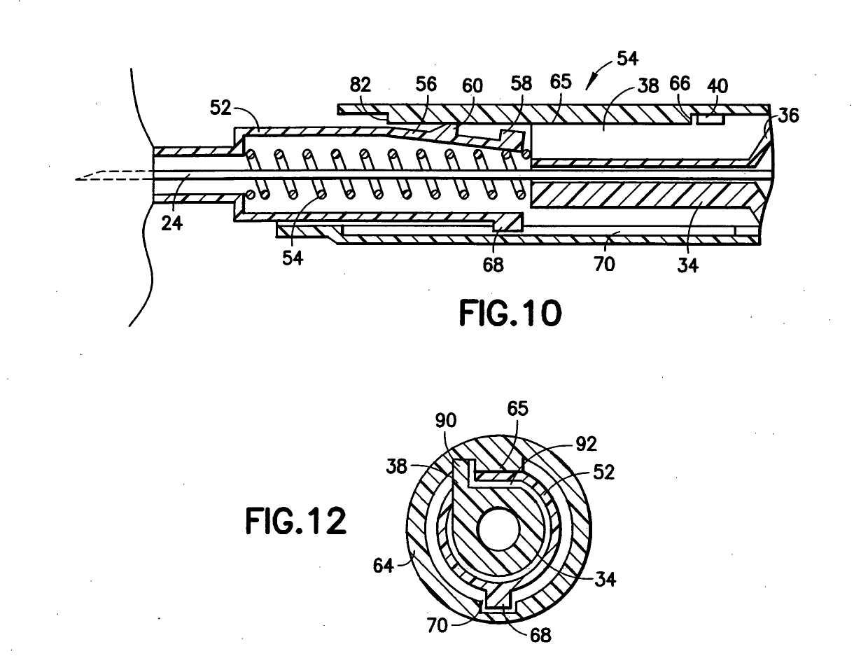

When medicament delivery pressure is applied, the needle cannula 24 is inserted in a patient for delivery of the medicament. Accordingly, when the shield 52 is initially activated, the shield 52 moves forward only until it contacts the patient's skin 88 as depicted in FIG. 10. After delivery of the dosage, the needle cannula 24 is withdrawn from the patient and the spring urges the shield 52 to the fully deployed position shown in FIG. 11 so that the needle cannula tip 27 is covered by the shield. In other words, once there is clearance for deployment of the shield—which occurs when the needle 24 is removed from the patient, the shield moves forward to cover the needled tip 27. The pin 68 of the shield 52 is guided in the track 70 until it reaches an end of the track 70 which prevents withdrawal of the shield 52 from the actuator 64. Furthermore, the retaining element 58 engages a locking face 82 on the actuator 64 when the shield is fully deployed which prevents the shield 52 from moving back toward the fully inserted position. Once use is complete, the device 10 can be disposed of in a sharps container.

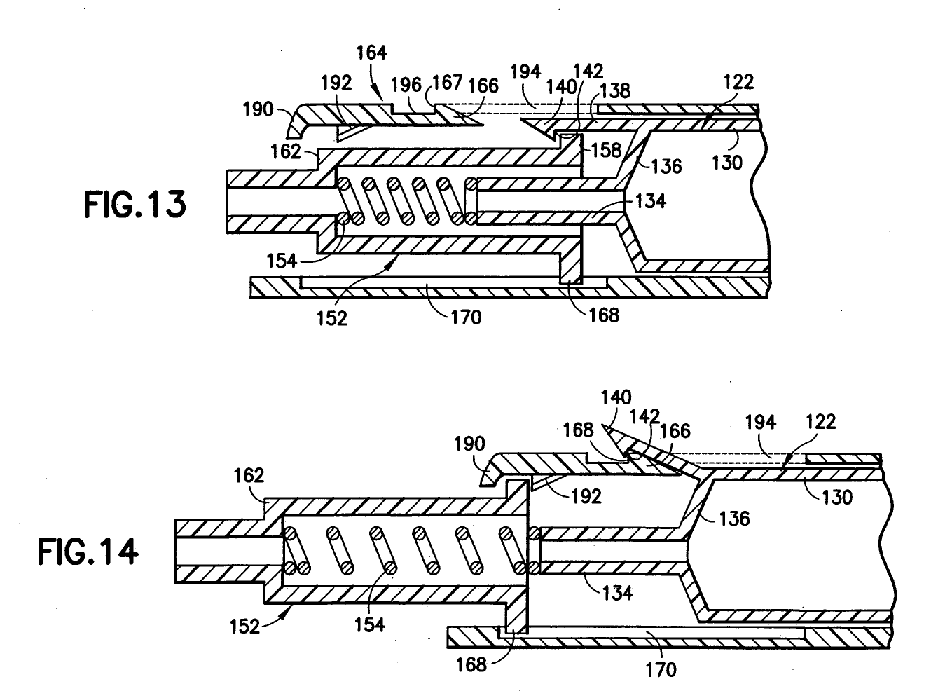

FIG. 13 shows an alternative embodiment of the medical device according to the present invention. A syringe barrel 122 includes a cylindrical portion 130 and a front portion including a small cylinder 134 and a transition section 136 between the cylindrical portion 130 and the small cylinder 134. The front portion of the syringe barrel 122 further includes a lever 138 with a retaining projection 140 arranged proximate a free end of the lever 138. A shield 152 is arranged on the front end of the syringe barrel 122. An urging member 154, such as a spring, is arranged between the small cylinder 134 and a step 162 on the shield 152 for urging the shield away from the syringe barrel 122. However, a catch 158 on the shield 152 engages the retaining projection 140, thereby preventing the shield from moving away from the syringe barrel 122 unless acted upon by an actuator 164 as explained below.

The actuator 164 is movably arranged on the barrel 122 and includes a release element 166. The actuator 164 also includes a slot 170 for receiving a pin 168 of the shield 152 and which guides movement of the shield 152. As in the previously described embodiment, the actuator 164 is moved from the location shown in FIG. 13 where catch 158 engages projection 140, to a retracted position shown in FIG. 14 when a medicament delivery force is applied to the medical device. During this movement, the release element 166 contacts the retaining projection 140 and forces the lever 138 to flex radially outward. The release element and retaining projection 140 have mutually inclined surfaces which slide against each other for this purpose. The actuator 164 is moved until retaining projection 140 rides completely over the release element 166 and is forced into a depression 196 in the outer surface of the actuator by the resilient return force stored in the lever 138, as shown in FIG. 14. As the retaining projection 140 is moved with the lever 138, the engagement of the retaining projection 140 and the catch 158 is released and the urgency of the spring 154 moves the shield 152 to an activated position shown in FIG. 14. In this position, the shield 152 covers the cannula needle tip 27 in a like manner as disclosed in the previously described embodiment (the needle cannula 24 is not shown in FIGS. 13-14).

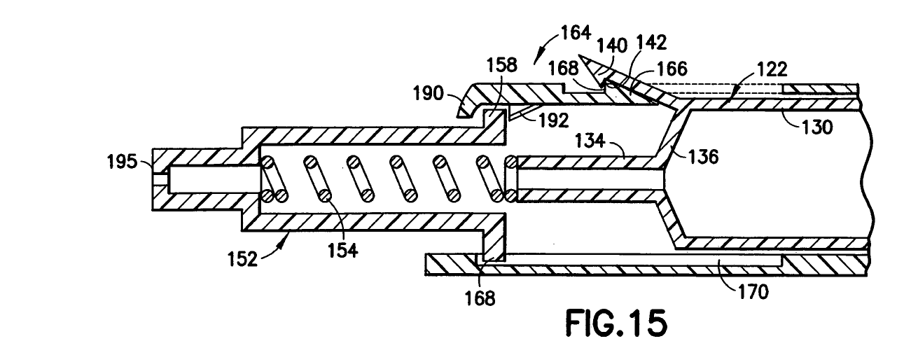

Forward movement of the shield 152 past the activated position is stopped by the interaction between the end of slot 170 and the pin 168. In addition, the actuator 164 may comprise a lip 190 for interacting with the catch 158. A retainer 192 may be arranged in the actuator 164 to prevent the shield 152 from moving back toward the syringe barrel 122. Alternatively, the lip 190 may be axially offset from the end of slot 170. When the shield 122 is pushed into the activated position, the pin 168 first contacts the end of slot 170 which causes the shield 122 to pivot about the initial point of contact at the end of slot 170 until the catch 158 rests on the lip 190 (or until the shield 122 contacts the cannula tip 27), as shown in FIG. 15. In this position, the shield 122 is askew relative to the cannula and the cannula is not aligned with the hole at the end of the shield 122 in which it was formerly inserted. This configuration prevents inadvertent sticks because when the shield 122 is pressed into the actuator 164, the tip of the needle is not aligned with the hole but, instead, contacts the step 162 or a wall 195 at the end of the shield 122 to maintain the needle tip within the shield.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements which perform substantially the same function in substantially the same way to achieve the same results are within the scope of the invention. Moreover, it should be recognized that structures and/or elements shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

1. A medical device for delivering a medicament to a patient, comprising:
a syringe assembly comprising:
a barrel having a forward end and a rear end and defining a reservoir within which the medicament may be contained;
a needle cannula having a forward tip and being coupled to said forward end of said barrel and in fluid communication with said reservoir; for delivering the medicament to the patient when said forward tip pierces the patient; and
a plunger having a first end with a stopper positioned in said reservoir and a second end having a thumb pad for receiving medicament delivery pressure for causing said plunger to move within said reservoir to cause the medicament to be expelled from said reservoir through said needle cannula forward tip;
a hollow shield body disposed on said front portion of said syringe barrel and releasably secured thereto, said shield body being selectively movable from a first position wherein said needle cannula forward tip is exposed, to a second position wherein said needle cannula forward tip is contained within said hollow shield body;
an urging member positioned between said hollow shield body and said barrel for imparting an urging force to said shield body for urging said shield body to said second position; and
an actuator mounted to said syringe barrel, said actuator axially movable from an initial position on said syringe barrel to a retracted position away from said needle cannula forward tip in response to the commencement of an application of the medicament delivery force to said thumb pad for releasing said hollow shield body from said barrel to allow said hollow shield body to move, under the urgency of said urging member, to said second position upon removal of said needle cannula tip from the patient.

2. The medical device of claim 1, wherein said syringe barrel is glass.

3. The medical device of claim 1, wherein said syringe barrel is plastic.

4. The medical device of claim 1, wherein said syringe barrel comprises a cylindrical barrel portion for holding the medicament and a front portion arranged proximate said front end of said syringe barrel for coupling to said cannula and said shield.

5. The medical device of claim 4, wherein said cylindrical barrel portion of said syringe barrel is glass and said front portion of said syringe barrel is plastic.

6. The medical device of claim 1, further comprising a retaining device formed on said hollow shield body for causing releasable securement of said hollow shield body to said front portion of said syringe barrel, said retaining device causing a release of said hollow shield body from said syringe barrel upon movement of said actuator to said retracted position.

7. The medical device of claim 6, wherein said retaining device comprises one of a catch element and a retaining element arranged on said front end of said syringe barrel, the other of said catch element and said retaining element being arranged on said hollow shield body, said catch element engaging said retaining element for preventing said hollow shield body from moving from said first position toward said second position.

8. The medical device of claim 7, wherein said retaining element comprises a flexible arm on said hollow shield body.

9. The medical device of claim 8, wherein said release mechanism comprises a portion of said actuator arranged to move said flexible arm to release said hollow shield body from said retention means when said actuator is moved to said retracted position.

10. The medical device of claim 9, wherein said portion of said actuator arranged to move said flexible arm comprises a cam arranged on an inner surface of said actuator.

11. The medical device of claim 10, wherein said cam further comprises a locking surface for engaging said hollow shield body when said hollow shield body is in the second position for preventing said hollow shield body from moving back toward the first position.

12. The medical device of claim 8, wherein said flexible arm is arranged on said hollow shield body and said catch is connected proximate said front end of said syringe barrel.

13. The medical device of claim 8, wherein the flexible arm is connected proximate said front end of said syringe barrel and said catch is arranged on said hollow shield body.

14. The medical device of claim 1, further comprising a first blocking device on said barrel for retaining said actuator in said retracted position.

15. The medical device of claim 14, wherein said first blocking device comprises an actuator catch arranged on said actuator and a projection on said syringe, barrel, said catch engaging said projection to retain said actuator in said retracted position.

16. The medical device of claim 6, further comprising a first blocking device on said barrel for retaining said actuator in said retracted position, and wherein said retaining device comprises a flexible arm having a projection arranged on said syringe barrel and a shield catch being arranged on said hollow shield body for engaging said projection when said shield is in said fully inserted position, said first blocking device comprising an actuator catch arranged on said actuator and an actuator projection arranged on said syringe barrel for engaging said actuator catch when said actuator is in said retracted position.

17. The medical device of claim 6, further comprising a first blocking device on said barrel for retaining said actuator in said retracted position, and wherein said retaining device comprises a shield catch arranged on said syringe barrel, a flexible arm having a projection arranged on said hollow shield body for engaging said shield catch when said shield is in said first position, said first blocking device comprising an actuator catch arranged on said actuator and an actuator projection arranged on said syringe barrel for engaging said actuator catch when said actuator is in said retracted position.

18. The medical device of claim 6, further comprising a first blocking device on said barrel for retaining said actuator in said retracted position, and a second blocking device for preventing movement of said actuator away from said retracted position when said actuator is in said initial position.

19. The medical device of claim 1, further comprising a removable clip connectable to said syringe barrel for preventing movement of said actuator to said retracted position while said clip is connected to said syringe barrel.

20. The medical device of claim 6, wherein said shield, said urging member, and said retention means are arranged in front of a medicament holding portion of said syringe barrel.

21. The medical device of claim 1, wherein said urging member device comprises a spring.

22. The medical device of claim 1, wherein said actuator further comprises a locking element for preventing movement of said hollow shield body from said second position back toward said first position.

23. The medical device of claim 1, wherein a front end of said actuator comprises a lip for retaining at least a portion of said shield at said second position when said shield is moved to said second position.

24. The medical device of claim 23, wherein said hollow shield body comprises a radially outward extending pin which is received in a slot defined in said actuator for guiding movement of said hollow shield body from said first position to said second position, wherein an end of said slot retains said shield at said second position when said hollow shield body is moved to said second position, wherein said end of said slot and said lip are axially offset such that said hollow shield body is misaligned with a longitudinal axis of said cannula when said hollow shield body is in said second position.

25. The medical device of claim 1, wherein said hollow shield body comprises a radially outward extending pin which is received in a slot defined in said actuator for guiding movement of said hollow shield body from said first position to said second position, wherein an end of said slot retains said hollow shield body at said second position when said hollow shield body is moved to said second position.

26. The medical device of claim 1, wherein said urging member comprises a coil spring.

27. The medical device of claim 1, wherein said actuator is moved to said retracted position in response to the application of the medicament delivery force before said plunger rod is moved into said syringe barrel for delivering the medicament.

28. A combination of a syringe assembly and a shield system;
wherein said syringe assembly comprises a barrel having a forward end and a rear end and defining a reservoir within which the medicament may be contained, a needle cannula having a forward tip and being coupled to said forward end of said barrel and in fluid communication with said reservoir; for delivering the medicament to the patient when said forward tip pierces the patient, and a plunger having a first end with a stopper positioned in said reservoir and a second end having a thumb pad for receiving medicament delivery pressure for causing said plunger to move within said reservoir to cause the medicament to be expelled from said reservoir through said needle cannula forward tip; and
wherein said shield system comprises a hollow shield body disposed on said front portion of said syringe barrel and releasably secured thereto, said shield body being selectively movable from a first position wherein said needle cannula forward tip is exposed, to a second position wherein said needle cannula forward tip is contained within said hollow shield body, an urging member positioned between said hollow shield body and said barrel for imparting an urging force to said shield body for urging said shield body to said second position, and an actuator mounted to said syringe barrel, said actuator axially movable from an initial position on said syringe barrel to a retracted position away from said needle cannula forward tip in response to the commencement of an application of the medicament delivery force to said thumb pad for releasing said hollow shield body from said barrel to allow said hollow shield body to move, under the urgency of said urging member, to said second position upon removal of said needle cannula tip from the patient.

29. The combination of claim 28, wherein said syringe barrel is glass.

30. The combination of claim 28, wherein said syringe barrel is plastic.

31. The combination of claim 28, wherein said syringe barrel comprises a cylindrical barrel portion for holding the medicament and a front portion arranged proximate said front end of said syringe barrel for coupling to said cannula and said hollow shield body.

32. The combination of claim 31, wherein said cylindrical barrel portion of said syringe barrel is glass and said front portion of said syringe barrel is plastic.

33. The combination of claim 28, wherein said shield assembly further comprises

34. The combination of claim 33, wherein said syringe barrel comprises one of a catch element and a retaining element arranged proximate a front end thereof and said retention device comprises the other of a catch element and a retaining element arranged on said shield, said catch element engaging said retaining element for preventing said shield from moving toward said second position when said shield is in said first position.

35. The combination of claim 34, wherein said one of a catch element and a retaining element comprises a catch element arranged on a web connected to said syringe barrel.

36. The combination of claim 34, wherein said one of a catch element and a retaining element comprises a retaining element arranged on a flexible arm connected proximate said front end of said syringe barrel.

37. The combination of claim 28, further comprising a first blocking device on said syringe barrel for retaining said actuator in said retracted position.

38. The combination of claim 37, wherein said first blocking device comprises a projection on said syringe barrel and said actuator comprises a catch engaging said projection to retain said actuator in said retracted position.

39. The combination of claim 38, further comprising a second blocking device on said syringe barrel for preventing movement of said actuator away from said retracted position when said actuator is in said initial position.

40. The combination of claim 28, further comprising a second blocking device on said syringe barrel for preventing movement of said actuator away from said retracted position when said actuator is in said initial position.

41. A medical device for delivering a medicament to a patient, comprising:
a syringe assembly comprising:
a barrel having a forward end and a rear end and defining a reservoir within which the medicament may be contained;
a needle cannula having a forward tip and being coupled to said forward end of said barrel and in fluid communication with said reservoir; for delivering the medicament to the patient when said forward tip pierces the patient; and
a plunger having a first end with a stopper positioned in said reservoir and a second end having a thumb pad for receiving medicament delivery pressure for causing said plunger to move within said reservoir to cause the medicament to be expelled from said reservoir through said needle cannula forward tip;
a hollow shield body disposed on said front portion of said syringe barrel and releasably secured thereto, said shield body being selectively movable from a first position wherein said needle cannula forward tip is exposed, to a second position wherein said needle cannula forward tip is contained within said hollow shield body;
means for urging said shield body from said first position to said second position;
means for releasably retaining said hollow shield body to said syringe barrel; and
means for actuating said retaining means for causing said urging means to move said hollow shield body to said second position, said actuating means being actuated upon the commencement of an application of the medicament delivery force to said thumb pad.
